(19) United States
(12) Patent Application Publication
(10) Pub. No.: US 2003/0017461 A1
(43) Pub. Date: Jan. 23, 2003

(54) Tag cleavage for detection of nucleic acids

(75) Inventors: Sharat Singh, San Jose, CA (US); Edwin F. Ullman, Atherton, CA (US); Bertram I. Rowland, Hillsborough, CA (US)

Correspondence Address: PERKINS COIE LLP, P.O. BOX 2168, MENLO PARK, CA, 94026, US

(73) Assignee: Aclara BioSciences, Inc.

(21) Appl. No.: 09/903,290

(22) Filed: Jul. 11, 2001

Related U.S. Application Data
(60) Provisional application No. 60/217,624, filed on Jul. 11, 2000.

(51) Int. Cl. C12Q 1/68; C12P 19/34

(52) U.S. Cl. 435/6; 435/91.2

(57) ABSTRACT

Methods and compositions are provided for nucleic acid analysis. The method employs a primer and a probe that bind to a target nucleic acid sequence, where the primer has an effector agent, which causes cleavage of a bond when the primer and the probe are bound to the same target molecule. The primer and probe have arms that do not bind to the target, hybridize with each other and comprise the effector and cleavable bond, where the probe has a tag defining the probe that is released upon bond cleavage. By having the probe complex at a lower Tₘ than the primer complex, the probe is released and can be cycled

This application claims the benefit of U.S. Provisional Application No. 60/217,624 filed Jul. 11, 2000, which is incorporated herewith by reference in its entirety.

### TECHNICAL FIELD

The field of this invention is the detection of nucleic acids, particularly in a multiplexed protocol.

### BACKGROUND

As the elucidation of the human genome and other genomes nears, there is increasing interest in how the information may be used to enhance medical practice. There is increasing evidence that the phenotype of an individual plays a crucial role in the health and well being of an individual. The ability to ward off disease, respond to infection, be resistant to cancer, neurological diseases, autoimmune diseases, and the like, appear to be intimately related to the phenotype. Inherited diseases, associated with mutations in a gene are well known and frequently occur at various phases during an individual's lifetime. There is also interest in relating phenotype to a repertoire of individual variations in the genotype. This requires that one have knowledge of the frequency with which a nucleotide is present at a specific site in a chromosome.

In many situations, particularly with single nucleotide polymorphisms (“snps”), there is an interest in making a plurality of determinations simultaneously. To this end, one wishes to have techniques that do not result in interference between the different components of the sample, provide for amplification of the nucleic acid sequences that are of interest without significant amplification of other nucleic acid sequences, permit miniaturization to minimize the amount of sample and reagents required for the determination, and the like.

There are a number of techniques for determining nucleic acid sequences and snps. One technique employs two nucleic acid sequences, a primer and a labeled probe, where the primer is extended in the presence of a cleavase. Extension of the primer results in cleaving the label from the probe. The Taqman® process employs a label involving a spaced apart fluorescer and quencher. When the correct sequence is present, the link between the fluorescer and quencher is cleaved, resulting in an increase in fluorescence. In many cases, the methods require the use of the polymerase chain reaction (“PCR”). While PCR is efficient in amplifying a few sequences in the same reaction, once the number of sequences to be amplified exceeds a relatively low level, the efficiency is substantially reduced.

There is, therefore, substantial interest in having protocols that are robust, allow for amplification of a large number of members of a sample without interference, and provide signals that are readily detected and can be individually identified.

### BRIEF DESCRIPTION OF THE PRIOR ART

References of interest include W097/740-28 AND 97/00967.

### SUMMARY OF THE INVENTION

Methods and compositions are provided for detecting nucleic acids, either a sequence of at least two nucleotides or a single nucleotide. The method employs a primer and a tagged probe, where the primer is bonded to a member of a bond cleaving system and the probe comprises a detectable tag bonded to a link susceptible to cleavage by said bond cleaving system. The primer and probe bind to a target nucleic acid, whereby the detectable tag is released. Desirably, the hybridization is performed at a temperature near the melting temperature of the complex of the probe with the target nucleic acid, so that the binding of the probe can be cycled with a plurality of detectable tags released for a single target molecule. The detectable tags may then be analyzed to determine the presence of the target nucleic acid. By employing detectable tags that can be individually analyzed, a plurality of nucleic acid moieties may be detected in the same sample medium.

### DESCRIPTION OF THE SPECIFIC EMBODIMENTS

Nucleic acid analysis is performed with a combination of a primer and a probe. The primer is characterized by having a nucleic acid binding sequence that binds to a target nucleic acid. Bonded to the sequence is a member of a bond-cleaving system. The probe comprises a nucleic acid binding sequence that hybridizes to a sequence of the target nucleic acid in proximity to the sequence to which the primer hybridizes. Bonded to the probe sequence is a detectable tag, which is bonded to a link susceptible to cleavage by the bond-cleaving system. Desirably, the melting temperature of the complex formed by the probe with the target nucleic acid is lower than the melting temperature of the complex formed by the primer with the target nucleic acid and the process is performed at a temperature at which the primer hybridization is substantially stable, while the primer hybridization, particularly after cleavage by the bond-cleaving system is substantially labile.

The method comprises combining under hybridizing conditions, the sample containing the target nucleic acid, and one or more pairs of primers and probes in a liquid medium. The temperature will depend upon whether cycling of the probe is intended, in which case the temperature will be between the melting temperature of the complexes of the template and probe, as influenced by the hybridization between the primer and probe, usually not more than about 10° C. greater than the melting temperature of the complex with the probe, conveniently not more than about 5° C. greater, and below the melting temperature of the primer and template. After sufficient time for hybridization and cleavage to occur, the liberated tags are analyzed in accordance with the nature of the tag.

The primer has two primary components joined by a link. The first component is a sequence that can hybridize to a target nucleic acid sequence. This sequence will have at least about 12 units (where a unit intends a base), more usually at least about 18 units and not more than about 100 units, usually not more than about 60 units and preferably not more than about 36 units. There will be a sufficient number of units to provide the desired affinity and specificity for the target and difference in binding affinity between the primer and probe. For the most part, the sequence will comprise naturally occurring bases, pyrimidines and purines, linked by natural or unnatural linkages, e.g. phosphate ribose or deoxyribose esters, phosphate ribose or deoxyribose thioesters, phosphate ribose or deoxyribose amides, amino acids, particularly glycine or alanine amides, where the base may be pendent from the amino nitrogen, or the like.

The primer also has an effector moiety, which is normally present as an arm that is not bound to the target sequence and comprises a member of a susceptible bond cleaving system. The effector moiety will usually be capable of cleaving a linkage between the detectable tag and the probe sequence in conjunction with other agents or may provide a complex that results in providing a labile linkage in the probe The probe sequence may be preprepared, so as to have the detectable tag bonded to the nucleic acid sequence when added to the reaction mixture or result from the addition of one or more nucleotides, usually one nucleotide, where the added nucleotide(s) includes the detectable tag linked to the nucleotide by means of a cleavable linker. The probe will be characterized by having a sequence, which hybridizes to a sequence of target nucleic acid in proximity to the sequence to which the primer binds. Usually, the probe and primer will be separated by fewer than six nucleotides along the chain of the target sequence, usually not more than three nucleotides and may be contiguous (0−6; 0−3). As indicated, the probe will ultimately have proximal to its terminus, either 3′ or 5′, the detectable tag linked through a cleavable linkage to the target nucleic acid binding sequence.

The primer will usually have at least the same number of nucleotides binding to the target sequence as the probe and usually more, generally at least about 10% more, more usually at least about 50% more and not more than about 5×, usually not more than about 3× the number of nucleotides. Generally the primer will have at least about 15 bases, more usually at least about 18 bases and not more than about 75 bases, generally not more than about 36 bases, binding to the target sequence. While the total number of bases above the indicated number for the primer is not critical, for the most part there will be no advantage to having a greater number of bases, and the larger the number of bases, the greater the cost.

The probe will also have two regions, the region binding to the target sequence and an arm that is not bound to the target sequence and comprises the cleavable linkage. The probe will have at least about 12 bases, usually at least about 15 bases and usually not more than about 36 bases binding to the target sequence. Desirably the probe will add to the specificity of the assay, when taken together with the primer, but will also allow for cycling. By performing the assay at a temperature between the melting temperature of the complex between the probe and the target nucleic acid and the melting temperature of the complex between the primer and the target nucleic acid, the primer will be substantially retained bound to the target nucleic acid, while the probe it will come on and off. During the probe's residence time bound to the target nucleic acid, the opportunity exists for the cleavable link to be cleaved by the effector while bound to the target nucleic acid. By providing for some binding energy between the unbound arms of the primer and probe, upon cleavage of the labile linkage, the melting temperature of the probe will be lowered allowing for greater release. In this manner, one may amplify the number of detectable tags that are released in relation to the number of target sequences present in the sample.

The bond-cleaving system may involve a variety of different components. One or more of the components may be on the arm of the primer. The primer associated component(s) may include an effector which may be an active participant in being involved in being a chemical component of a chemical reaction or may be a passive participant, providing an environment recognized by an enzyme, where the enzyme will act as a hydrolase, or both. The effector normally will cooperate with another agent that may be a chemical or physical agent, such as electromagnetic radiation or the like. Therefore, the effector will normally be unchanged or cycled. The effector may be organic or inorganic, if inorganic, usually metalloorganic, will usually require additional agents, either physical or chemical. Included among the effectors are enzymes, ribozymes, metalloorganic compounds, electromagnetic radiation sensitizers, nucleic acid sequences, or other agents that can be cycled and provide, directly or indirectly, for cleavage of a linkage.

Various enzymes may be used, where the enzymes can cleave one strand of a hybrid DNA-RNA strand, a uracil, a glycol linkage, where the glycol linkage may take other forms than a ribose or deoxyribose diphosphate. The enzymes may be bound to the primer, covalently or non-covalently, or free in solution. A number of enzymes are known that selectively cleave one of the strands of the hybrid complex. These enzymes include RNase H, Rnase Hi and like enzymes from a variety of species, as described in Cerritelli and Crouch, 1998 Genomics 53, 300-307, and references cited therein. Enzymes that cleave at apurinic and apyrimidinic sites in dsDNA include AP endonucleases, as described in Doetsch and Cunningham, 1990 Mutation Research 236, 173-201; and Levin and Demple, 1990 Nucleic Acids Res. 18, 5069-5075. Enzymes are available that cleave a DNA strand comprising a uracil, such as uracil-DNA DNA glycosylase, as described in Weiss et al., 1983 Biochemistry 22, 4501-4507. Restriction enzymes that cleave at only one strand include site specific nickases, as described in Abdurashitov, et al., 1996 Molecular Biology 30, 754-758. Other enzymes will cleave at a loop, as a result of a mismatch forming a loop out. Such enzymes include resolvases and specific endonucleases, as described in Tito, jr., et al., 1998 Clin. Chem. 44, 731-739 and Babon, et al., 1999 Electrophoresis 20,1162-1170. Other enzymes are specific for a particular functionality, such as peroxidase for a peroxide, sulfatase for a sulfate ester, pyrophosphatase for pyrophosphate, cholinesterase for acetylcholine, β-galactosidase for β-galactosidyl ethers, and the like. Thus, linkages involving peroxide groups, ester groups, anhydride groups, ether groups, as well as other functional groups may serve as the linkage for cleavage.

Instead of having a moiety resulting in complex recognition by an enzyme, one may have a cofactor bonded to the primer. By providing for cycling of the cofactor, so that the proper oxidation state of the cofactor is restored after the enzymatic reaction, the signal from a single target molecule can be amplified as described above. Various cofactors include NAD, NADP, FAD, ascorbate, glutathione, etc. and the reduced analogs thereof, when used in conjunction with metal ions such as copper or iron, as described in Oikawa and Kawanishi, 1996 Biochemistry 35, 4584-4590; and Dreyer and Dervan,1985 PNAS USA, 82, 968-972.

Another system employs electromagnetic radiation sensitizers, where the moiety bonded to the primer transfers energy to the linkage with resulting cleavage of the linkage. For the most part, the energy transfer moieties are dyes absorbing light in the range of about 275 to 600 nm, more usually in the range of about 275 to 350 nm. Of interest are intercalating dyes bound to the primer, such as porphyrins, as described in Munson and Fiel, 1992 Nucleic Acids Res. 20, 1315-1319. Illustrative dyes include texaphrin and metal complexed texaphrin (U.S. Pat. Nos. 5,714,328 and 5,798,491), diazapyrene (U.S. Pat. No. 4,925,937), etc., where the labile bonds cleaved by energy transfer include peroxide, e.g. oxetane, alpha, beta-dicarbonyl, o-ntirobenzyloxy, hydrindanyloxy, etc. Illustrative dyes also include texaphrin and metal complexed texaphrin (U.S. Pat. Nos. 5,714,328 and 5,798,491), diazapyrene (U.S. Pat. No. 4,925,937), etc., where the labile bonds cleaved by energy transfer include peroxide, e.g. oxetane, alpha, beta-dicarbonyl, o-ntirobenzyloxy, hydrindanyloxy, etc.

The effector molecule may be bonded to the primer nucleic acid sequence by any convenient linking group. The length of the linking group will depend on the nature of the effector molecule, the nature of the cleavable linkage, synthetic convenience, and the like. In many instances, the effector molecule may be added to the end of the synthesized nucleic acid sequence, during the synthesis of the sequence. This will usually be possible with smaller effector molecules, under about 5,000 Dal. For larger molecules, such as enzymes, the nucleic acid will usually be added to the enzyme after the nucleic acid has been synthesized. By providing for a functionality that will react with a functionality present on the enzyme. Depending on the amino acids present in the enzyme and the nature of the active site, various strategies may be used for the conjugation. Where one or more lysines are available, one may use maleic anhydride to form the maleimide, which may then be conjugated with a thiol to form the thioether. Where a cysteine is present on the enzyme, one may use active halogen, such as a benzyl halide for conjugation to form a thioether. With lysines present, one may use reductive amination with an aldehyde in the presence of a hydride supplying reductant. Techniques for conjugating proteins to nucleic acids are well known in the literature. See, for example, W099/41273.

Similarly, the tag through the cleavable linker may be bonded to the probe sequence. The tags can be conveniently linked to the terminal nucleotide, either prior to or subsequent to the addition of the terminal nucleotide during synthesis. Various techniques are described in the literature to provide substituents at the terminal position. Alternatively, the linkage of the synthetic sequence to the solid support can provide for a functional group that will be available upon cleavage from the solid support. The functional group may then be used for linking the tag through the cleavable linkage. In this way, amides, esters, thioethers, or ethers can be employed for linking the tag and the probe sequence. Conveniently, the active functional groups will be protected during synthesis and these groups may be retained while the tag is conjugated to the sequence, after which they may be removed.

A tag molecule is one that can be differentiated from other tag molecules to allow for multiple target sequences to be analyzed in the same sample. The tag molecules are conveniently differentiated by their mobility in electrophoresis. They may have one or more bases for differentiation, where one of the bases will be mobility and a second basis may be a different detectable signal, such as electromagnetic radiation, as observed with fluorescence, chemiluminescence, electrochemiluminescence, electrical signal, etc. In this manner, the different tags will be unique as to the two or more differences, so that two tags may have the some mobility, if they may be distinguished by their fluorescence. The tags have three characteristics, which may be more or less associated with different regions of the tag. A tag will have mass, charge and be capable of detection. The mass may come from neutral molecules, charged molecules or combinations thereof. The portion of the tag associated with the mass may provide properties other than mass, such as solubility, enhancing the signal by providing for a propitious environment, allow for easy handling, synthesis or the like. Mass enhancing entities may be a single molecule without a pattern of repeating groups or have a repeating group, where the repeating group will be the individual unit of mass. The repeating unit may be a divalent group, such as a hydrocarbylene or substituted hydrocarbylene, such as alkylene, e.g. methylene, ethylenene, propylene, etc., having neutral substituents, such as ethers, thioethers, hydroxyl, thiols, esters, both organic and inorganic, cyano, nitro, halo, etc., may be hydrocarbylene separated by a neutral heteroatom, e.g. oxygen, sulfur, acylamido, where the nitrogen may be the intervening group or carbamyl may be the intervening group, phosphate as the triester, etc. The hydrocarbylene will be aliphatic, alicyclic, or aromatic. Alternatively, the repeating unit may be a divalent heterocyclic group, having from 1 to 3 annular heteroatoms, which are O, S, N, or P and may be combined with hydrocarbylene. The neutral mass providing entity will generally range from about 2 to 60, more usually 2 to 36 carbon atoms and from about 0 to 8 heteroatoms for the substituted hydrocarbylene or heterocyclic groups, and will usually have from 1 to 4, more usually 1 to 3 heteroatoms as a linking unit between repeating carbon containing units. The heteroatom links may be the same or different in the chain, usually the same. The groups may be polar or non-polar, preferably polar imparting water solubility.

Groups of interest may include alkylene groups, alkyleneoxy groups, alkylenethio groups, alkylenesulfone groups, glycineamide, alanineamide, serineamide, methionineamide, copolymer of ethylene diamine and succinic acid, copolymer of ethylene glycol and fumaric acid, and the like. The mass enhancing moiety will be selected based on considerations of covenience, stability under the conditions of the determination, synthetic convenience, interactions with other components of the tag and of the determination, and the like.

The charge enhancing moiety may be a single molecule or an oligomer of the same or different units, usually have the same type of linking group. Thus, the chain may be of the same nature as the mass enhancing moiety, except that the units will contribute charges to the moiety. The charges may be positive or negative. For the negative groups, illustrative groups are acyl groups, both organic and inorganic, such as carboxyl, sulfonate, sulfinate, phosphate, phosphinate, borate, phenolic hydroxyl, etc. For the positive groups, illustrative groups are amine and ammonium, sulfonium, phosphonium, metal chelates, metallocenes, etc. For them most part, oligopeptides are convenient, providing amine groups as in lysine, arginine and histidine and carboxyl groups as in aspartate and glutamate, and these will be preferred. Alternatively, alkylene imines and oligomers thereof may be used, particularly N-alkylated imines. Depending on the nature of the moiety, there may be one or two neutral units between charged units. Generally, the number of charges will be in the range of about 1 to 10, usually 1 to 6, and frequently in the range of 1 to 4. The moiety will usually have from about 2 to 60 carbon atoms, more usually 2 to 30 carbon atoms and at least one heteroatom and may have as many as 30 heteroatoms, usually not more than about 20 heteroatoms associated with the functional groups indicated above.

The detectable moiety will generally be detectable by electromagnetic radiation, e.g. luminescent, or electrically, by its redox potential. For the former, the label may be fluorescent, chemiluminescent or phosphorescent, particularly fluorescent. For the electrically detectable label, the label will usually be an organometallic compound.

The tag will generally have a molecular weight in the range of about 100 Dal to 5 kDal, depending on the number of tags necessary for the determination. The more tags required for analysis, the larger will be the range in molecular weight.

The tags may be prepared by conventional synthetic techniques. The tag may be preprepared and attached to the nucleic acid sequence or be added to the sequence in the process of synthesizing the nucleic acid sequence on a support. Depending on the nature of the cleavable linkage, the cleavable linkage may be attached to either the nucleic acid sequence or the tag, prior to joining the tag to the nucleic acid sequence.

The target sequence may come from a variety of sources, both prokaryote and eukaryote, unicellular and multicellular organisms, mammalian and plant, etc. The target nucleic acid may be RNA or DNA, where the nucleic acid may be mRNA, tRNA, cDNA, chromosomal DNA, plastid DNA, mitochondrial DNA, etc., where the DNA may be direct from the host, reverse transcribed from RNA, amplified with PCR or other amplification scheme, or the like. Depending on the source of the DNA, the DNA may be subject to further processing, such as desalting, chromatography, separation into fractions, e.g. nucleosomes, mitochondrial DNA, nucleolar DNA, chromsomal DNA or individual chromosomes, etc. Desirably the fragments will be less than about 1 cM, preferably less than about 0.1 cM and usually at least about 200 nt, more usually at least about 300 nt, usually single stranded. The DNA will be dispersed in an appropriate hybridization medium, generally having a conventional buffer, depending upon the nature of the effector. Buffers include phosphate, borate, HEPES, MOPS, acetate, etc. The buffer concentration will generally be in the range of about 10 to 200 mM, more usually in the amount of 50 to 200 mM. Other salts may be present to enhance the stringency, such as sodium chloride, sodium dodecyl sulfate, potassium chloride, magnesium chloride, etc., generally present in the range of about 0 to 200 mM. For enhancing stringency, small amounts of non-interfering water-soluble organic solvents may be included, such as methanol, ethanol, acetonitrile, hexamethylphosphoramide, dimethylformamide, etc, generally being present in the aqueous solution in less than about 20 volume %, usually less than about 10 volume %. For the most part, to enhance stringency, elevated temperatures will be used, by itself or in conjunction with one or more of the other stringency-enhancing agents.

The different agents are combined with the sample in an appropriate medium as described above at a temperature, usually in the range of about 100 to 60° C., more usually in the range of about 200 to 50° C. The temperature will be selected in accordance with the melting temperatures of the primer and the probe, the stability of the enzyme, when an enzyme is present, the desired rate of cycling, the necessary residence time of the probe in order to have a reasonable rate of reaction, generally in the range of about 1 min to 6 h, usually in the range of about 5min to 3 h and preferably in the range of about 5min to 1 h. Where enzyme is labile under the conditions of the reaction, additional reagent may be added at intervals to maintain the reaction rate. Where one is only interested in a qualitative answer, it is only necessary that the reaction occur to provide a detectable amount of product and the amount of enzyme and time are not critical.

The concentration of the DNA sample will generally be in the range of about 1 pM to 1 mM, more usually in the range of about 1 pM to 1 microM. The primer and probe will generally be at least about equal to and usually in excess of the anticipated concentration range of the target DNA. The primer will be present in a mole ratio of about 1-10:1, more usually 1-5:1, while the probe will usually be in a mole ratio of 1-500:1, usually in the range of about 10-200:1. The important factor for the primer is that most, if not all, of the target sequence present will be bound by the primer. For the probe, one will usually want an excess to allow for amplification. However, where the probe can undergo cleavage in the absence of being bound to the target DNA, the greater the amount of probe present, the greater the background. Therefore, the optimal amount of probe will vary with the nature of the probe and the amount of background reaction as compared to reaction when the probe is bound.

There will be at least one target sequence to be measured, more usually at least 2. Preferably, the number of target sequences to be measured will be at least about 10 and may be a 1,000 or more, usually not more than about 500, more usually not more than about 200. For each target sequence, one primer and one probe will be added to the assay mixture. In addition, all the reagents necessary for releasing the tag from the nucleic acid sequence will also be added. Depending on the nature of effector system, the temperature may be thermally cycled or maintained during the course of the reaction. Generally the temperature necessary for amplification of the signal will be at least about 35° C., more usually and least about 45° C. and not more than about 80° C., usually not more than about 65° C. The reaction time will be sufficient for at least a sufficient amount of tag to be released for detection, usually requiring at least one minute, more usually requiring at least about 5 min and not more than about 24 h, usually not more than about 12 h and preferably not more than about 6 h.

After the reaction is complete, the assay mixture may be analyzed. The assay mixture may be concentrated, diluted or be unchanged when added to the capillary electrophoretic analytical device. In some instances, one may wish to separate the tags from the other material present in the assay mixture. Depending on the nature of the tags, the tags may be separated by precipitation of the tags or the nucleic acid, chromatography, extraction, or the like. The liquid medium enriched for the tags may then be used for analysis. Usually, the electrophoresis will be performed with a sieving medium that provides for clean separation of the tags. The conditions for performing the electrophoresis will vary with the nature of the tags and will be evident to those of skill in the art.

Alternatively, one may use liquid chromatography to separate the tags, the different mass/charge ratios providing for different mobility. The packing of the channel may provide for ion exchange as well as sieving through the column. Depending on the density of the packing various methods of providing for flow through the column may be employed. One may use electroosmosis for a lightly packed column or pumping or applied pressure for more densely packed columns.

In order to minimize the release of the tags adventitiously from the probe, various protective mechanisms may be employed. One mechanism involves using a stem and loop structure, where the stem provides protection for the labile bond. The labile bond can be in one of the strands involved in the stem. In those situations, where cleavage is as a result of DNA-RNA hybridization, the stem may involve two RNA or two DNA strands, which would not be recognized by the nuclease. Where the cleavage is as a result of a mismatch, the two strands of the stem would be complementary. Alternatively, where the bond is chemically scissile, the presence of the other strand would inhibit chemical attack. In the event of a photolytically labile bond, subject to cleavage in the presence of a sensitizer, the other strand would provide for light absorption without energy transfer, so as to protect the photolytically labile bond. The stem need not be very long, sufficiently ling to be reasonably stable under the conditions of the assay, but resulting in a more stable binding when the loop, and potentially a portion of one strand of the stem is bound to the target sequence. By having at least a portion of the probe in the stem and loop conformation, the potential for false positives will be diminished. The particular length of the stem, its melting temperature and the choice of nucleotides may be determined empirically in light of the mechanism of cleavage, the temperature at which the determination is made, and the like.

The following examples are offered by way of illustration and not by way of limitation.

Experimental

### EXAMPLE 1

### Singleplex SNP Detection using a CviNY2A Nickase Reaction

| | |
|---|---|
| Target: | . . . CGCAA TAGCC TAGCA GT(c)GC . . . |
| | . . . GCGTT ATCGG ATCGT CA(g)CG . . . |
| Primer: | 5′-CGCAATAGCCTAGCCACTTG-3′ |
| Probe: | *ACLA001-5′-CAAGTCGTcGC-3′ |
| | \hat{ } |

* eTag fluorescent group: Fluorescein(C₆P)₁ (ACLA001)
\hat{ } indicates site of cleavage
c indicates base complementary to SNP locus

10 pmole primer is mixed with 100 ng template DNA to a final volume of 10 μl in Nickase buffer composed of 10 mM Tris-HCl, pH 8.3,10 mM MgCl₂, 150 mM KCl. The sample is heated to 95° C. for 5 min, is then annealed by cooling to 42° C. for 15 min, followed by 15 min at 30° C. 1 μl of annealing reaction is combined with 1 nmol of probe and 0.1 units of CviNY2A in Nickase buffer plus 100 μg/ml BSA to a final volume of 10 μl. The sample is incubated at 30° C. for 30 min, then heated to 65° C. for 10 min to inactivate enzyme.

The released electrophoretic tag is separated from undegraded probe using a μicrochannel device described in detail in PN 5,900,130, and having a configuration generally as sketched in FIG. 1a of PN 5,900,130. Briefly, this microchannel device is constructed by forming a base plate and cover of acrylic polymer, with apposing a surfaces bonded together by thermobonding. The microchannel structure corresponds to two crossed linear channels of dimensions 8 mm and 5.2 cm in length. The channel has a trapezoidal cross-section, measuring at widest about 80 μm and at narrowest about 30 μm, with an average depth about 20 μm. At the termini of the channels, holes of 2 mm in diameter are drilled as buffer reservoirs. Platinum electrodes are introduced into each of the four reservoirs.

The assembled device is loaded with buffer by filling reservoirs 1, 2, and 4 with buffer and then applying a vacuum at reservoir 3 to draw the buffer into the channels 11, 12, 13, 14; then reservoir 3 is filled with buffer. Buffer is removed from sample supply reservoir 4, and replaced with 5 μl of sample material. Voltages are applied in stages as indicated in the table below, yielding movement of sample as illustrated in FIGS. 1a, b and 4a, b of PN 5,900,130. The “injection” stage draws sample from reservoir 4 towards reservoir 2, with the borders of the sample stream constrained by buffer drawn from reservoirs 1 and 3 towards reservoir 2. Voltages are then switched to the “separation” stage settings, causing a unit of the sample stream located in the microchannel intersection to enter channel 13, migrating towards reservoir 3, and causing remaining sample in the stream bordering the sample unit to be pulled away from the channel intersection and back towards reservoirs 2 and 4. Components of the sample are separated in channel 13 according to their electrophoretic mobilities, then detected by fluorescence using an argon-ion laser operating at 488 nm to excite the fluorescein, and a photomultiplier tube with emission filter for detection at 520 nm.

| Running | | | | | | |
|---|---|---|---|---|---|---|
| conditions: | 1 | 2 | 3 | 4 | time | |
| Injection | 365 V | 730 V | 0 | 365 V | 60 s | 100 V/cm |
| Separation | 0 | 300 V | 1420 V | 280 V | 500 s | 250 V/cm |

### EXAMPLE 2

Multiplexed SNP Detection using an ExoIII Exonu-
clease Reaction

Target #1:

| | |
|---|---|
| Target: | . . . CTAGCAGT(c/g) G CAGGATACTG ACCTGCGC . . . |
| | . . . GATCGTCA(g/c) C GTCCTATGAC TGGACGCG . . . |
| Primer: | 5′-CAAGTCGGAT ACTGACCTGC AT-3′ |
| Probes: | 5′-GTcGCCACTT G-3′-ACLA001* |
| | \hat{ } |
| | 5′-GTgGCCACTT G-3′-ACLA016* |
| | \hat{ } |

Target #2:

| | |
|---|---|
| Target: | . . . GAGATCCA(c/g)CAGCA AAGTC CGAGT CGGT . . . |
| | . . . CTCTAGGT(g/c)GTCGT TTCAG GCTCA GCCA . . . |
| Primer: | 5′-CAAGTACCAA AGTCCGAGTC GAA-3′ |
| Probes: | 5′-CAcCACTACT TG-3′-ACLA018* |
| | \hat{ } |
| | 5′-CAgCACTACT TG-3′-ACLA019* |
| | \hat{ } |

* indicates eTag fluorescent groups (all containing Fluorescein):
ACLA001: Fl(C₆P)₁-oligonucleotide
ACLA016: Fl(C₆P)₁(C₃P)₁-oligonucleotide
ACLA018: Fl(C₆P)₁(C₃P)₂-oligonucleotide
ACLA019: Fl(C₆P)₁(C₃P)₃-oligonucleotide
\hat{ } indicates site of cleavage
c or g indicates base complimentary to SNP locus

10 pmole of each primer is mixed with 100 ng template DNA to a final volume of 10 μl in exonuclease buffer composed of 66 mM Tris-HCl, pH 8.3 and 6.6 mM MgCl₂. The sample is heated to 95° C. for 5 min, then annealed by cooling to 42° C. for 15 min, followed by 15 min at 30° C. 1 μl of annealing reaction is combined with 1 nmol of each of the four probes and 0.1 units of Exolll in exonuclease buffer to a final volume of 10 μl. The sample is incubated at 30° C. for 30 min, then heated to 65° C. for 10 min to inactivate enzyme.

The released electrophoretic tag is separated from undegraded probe using a μicrochannel device as described in Example 1. The assembled device is loaded with buffer followed by 5 μl of sample material as described above. Voltages are applied in stages as indicated in the table below, yielding movement of sample as illustrated in FIGS. 1a, b and 4a, b of PN 5,900,130. Components of the sample are separated in channel 13 according to their electrophoretic mobilities, then detected as described in Example 1.

| Running conditions: | 1 | 2 | 3 | 4 | time |
|---|---|---|---|---|---|
| Injection | 550 V | 1100 V | 0 | 550 V | 60 s |
| Separation | 0 | 450 V | 2130 V | 420 V | 500 s |

It is evident from the above description and examples that the subject method provides for an efficient method for identifying nucleic acid sequences or one or a few nucleotides in a nucleic acid sequence. The methodology provides for multiplexing, so that a single sample may be interrogated as to one or numerous aspects of the sample. In this way, analyses, such as single nucleotide polymorphisms, mutations, expression profiles, etc., may be analyzed in a single medium and the results determined by separating the tags. In addition, by appropriate choice of conditions, the signal may be amplified, so that a single target results in a plurality of detectable products.

All publications and patent applications mentioned in this specification are indicative of the level of skill of those skilled in the art to which this invention pertains. All publications and patent applications set forth herein are incorporated by reference to the same extent as if each individual publication or patent application was specifically and individually indicated to be incorporate by reference.

The invention now having been fully described, it will be apparent to one of ordinary skill in the art that many changes and modifications can be made thereto without departing from the spirit or scope of the appended claims.

It is claimed:

1. A method for detecting at least one nucleic acid target sequence in a nucleic acid sample, employing an effector system capable of cleaving a labile linkage, a primer and a probe for each target sequence, wherein for each target sequence, said primer comprises a first sequence that hybridizes to a first portion of said target sequence and a member of said effector system, and said probe comprises a second sequence that hybridizes to a second portion of said target sequence proximal to said first portion and a tag specific to the target sequence linked to said second sequence through said labile linkage, said method comprising:
combining under hybridizing conditions, said sample, said primer and said probe and any additional members of said effector system under conditions where said primer substantially stably binds to said first sequence and said probe reversibly binds to said second sequence, whereby when said primer and probe are both bound to said target sequence said labile linkage is cleaved by said effector system releasing said tag bound to said probe; and
analyzing for released tags as related to the presence of said target sequence.

2. A method according to claim 1, wherein one of said member and said labile linkage is DNA and the other is RNA and said effector system comprises an enzyme which cleaves the labile linkage when said labile linkage is hybridized to said member.

3. A method according to claim 1, wherein said member comprises a metalloorganic moiety and said labile linkage is oxidatively labile, where when said labile linkage is cleaved said metalloorganic moiety is reduced, and said effector system comprises an electron receptor for cycling said metalloorganic moiety.

4. A method according to claim 1, wherein said member is an enzyme and said labile linkage is cleaved by said enzyme.
